(19) United States
(12) Patent Application Publication
(10) Pub. No.: US 2026/0115163 A1
(43) Pub. Date: Apr. 30, 2026

(54) NOVEL TREATMENTS

(71) Applicant: 3Z EHF, Reykjavik (IS)

(72) Inventors: Karl Ægir KARLSSON, Reykjavik (IS); Haraldur THORSTEINSSON, Reykjavik (IS)

(73) Assignee: 3Z EHF, Reykjavik (IS)

(21) Appl. No.: 19/427,233

(22) Filed: Dec. 19, 2025

Related U.S. Application Data
(63) Continuation-in-part of application No. 19/247,183, filed on Jun. 24, 2025.
Application No. 19/247,183 is a continuation of application No. 18/274,945, filed on Jul. 28, 2023.

(30) Foreign Application Priority Data
Jan. 29, 2021 (GB) GB2101291.9
Jan. 29, 2021 (GB) GB2101292.7
Jan. 29, 2021 (GB) GB2101296.8
Jan. 28, 2022 (WO) PCT/EP2022/052142

(51) Int. Cl. A61K 31/216; A61K 31/4422; A61K 31/498; A61P 25/00

(52) U.S. Cl. CPC A61K 31/216; A61K 31/4422; A61K 31/498; A61P 25/00

(57) ABSTRACT

The present invention provides aceclofenac, amlodipine or doxazosin, or a pharmaceutically acceptable salt or solvate thereof, for use in the treatment or prophylaxis of Attention Deficit/Hyperactivity Disorder (ADHD). The invention also provides kits and methods for the treatment or prophylaxis of ADHD.

### CROSS-REFERENCE TO RELATED APPLICATIONS

The present application is a continuation-in-part of U.S. patent application Ser. No. 19/247,183, filed Jun. 24, 2025, which is a continuation of U.S. patent application Ser. No. 18/274,945, filed Jul. 28, 2023, which is a U.S. National Stage of PCT International Patent Application No. PCT/EP2022/052142 filed Jan. 28, 2022, which claims priority to Application Nos. GB2101291.9, filed Jan. 29, 2021, GB2101292.7, filed Jan. 29, 2021, and GB2101296.8, filed Jan. 29, 2021.

### FIELD OF THE INVENTION

The present invention relates to the treatment and/or prophylaxis of Attention Deficit/Hyperactivity Disorder (ADHD). The present invention also relates to dosage regimens and kits that find utility in the treatment and/or prophylaxis of ADHD.

### BACKGROUND OF THE INVENTION

Attention Deficit/Hyperactivity Disorder (ADHD) is a prevalent, heterogeneous neurodevelopmental disorder characterised by impaired levels of attention, hyperactivity or impulsivity, or a combination thereof. It is estimated that the condition affects approximately 5% of individuals under the age of 18 years worldwide with persistence of symptoms into adulthood in approximately 65% of cases, causing considerable functional impairment and comorbidity across the lifespan. The prevalence of ADHD in adults is estimated to be approximately 2.5% worldwide (Thapar & Cooper, The Lancet, 2016, 387(10024), 1240-1250).

The aetiology of ADHD is complex and not fully understood. However, impairment of dopaminergic neurotransmission is considered to be a common feature in ADHD patients. Recent studies have also found that genetic mutations in the Latrophilin 3 (LPHN3, also referred to as ADGRL3) gene are strongly associated with ADHD (Arcos-Burgos et al., 2010, Molecular Psychiatry, 15, 1053-1066). Further studies in zebrafish have shown that down regulation of latrophihn3.1 (lphn3.1), the zebrafish LPHN3 homologue, results in hyperactivity (Lange et al. Mol Psychiatry 2012, 17, 946-954 and Lange et al., 2018, Prog Neuropsychopharm Biol Psych, 84, 181-189). These studies also suggest that down regulation of lphn3.1 is linked to aberrant dopaminergic neurotransmission.

Established treatments for ADHD include pharmacological treatments such as stimulants (for example, methylphenidate, dexamphetamine, and lisdexamfetamine), norepinephrine reuptake inhibitors (for example, atomoxetine) and α2-adrenergic agonists (for example, guanfacine and clonidine). Treatment of ADHD may also include the use of non-pharmacological therapies either as a monotherapy or in combination with pharmacological treatments. Examples of non-pharmacological treatments that may benefit patients with ADHD include cognitive behavioural therapy (CBT), dietary treatments (for example, supplementary fatty acids and the exclusion of artificial food colour), and exercise programs.

Clinical guidelines such as the UK National Institute for Health and Care Excellence (NICE) guidelines recommend the use of methylphenidate, atomoxetine, and dexamphetamine for the treatment of ADHD in children or adolescents, and lisdexamfetamine or methylphenidate for the treatment of ADHD in adults. However, despite the approved use of such treatments for ADHD, their use remains controversial due to their limited efficacy and/or adverse effects (Cortese et al., 2018, The Lancet, Psychiatry, 5(9), 727-738). These medications can have significant drawbacks. Stimulants often cause decreased appetite, headaches, hypertension, and sleep disturbances, and carry a high abuse risk. Non-stimulants can often show lower efficacy and cause fatigue, somnolence, agitation, and aggression. Approximately 25% of patients fail to respond to these medications, highlighting the need for well-tolerated and mechanistically distinct treatment options.

Thus, there remains a need for further treatments for ADHD that provide clinical benefits whilst also displaying good clinical tolerability.

### SUMMARY OF THE INVENTION

The present invention provides a compound according to formula (I)

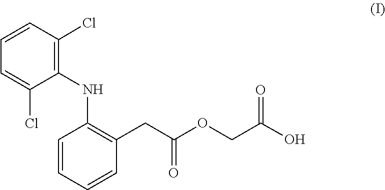 [SMILES: O=C(O)COC(=O)Cc1ccccc1Nc1c(Cl)cccc1Cl]

or a pharmaceutically acceptable salt or solvate thereof; for use in the treatment or prophylaxis of Attention Deficit/Hyperactivity Disorder (ADHD).

The present invention also provides a compound according to formula (II)

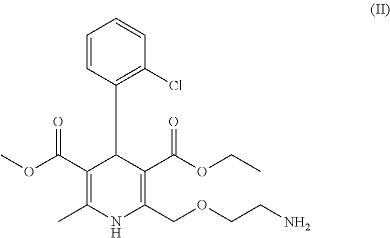 [SMILES: CCOC(=O)C1=C(COCCN)NC(C)=C(C(=O)OC)C1c1ccccc1Cl]

or a pharmaceutically acceptable salt or solvate thereof; for use in the treatment or prophylaxis of Attention Deficit/Hyperactivity Disorder (ADHD).

The present invention also provides a compound according to formula (I) in the S-enantiomer configuration

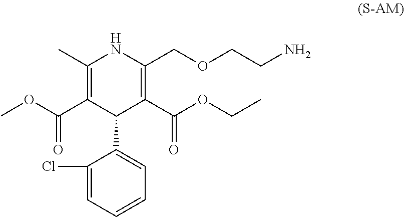 [SMILES: CCOC(=O)C1=C(COCCN)NC(C)=C(C(=O)OC)[C@H]1c1ccccc1Cl]

or a pharmaceutically acceptable salt or solvate thereof; for use in the treatment or prophylaxis of Attention Deficit/Hyperactivity Disorder (ADHD).

The present invention also provides a compound according to formula (III)

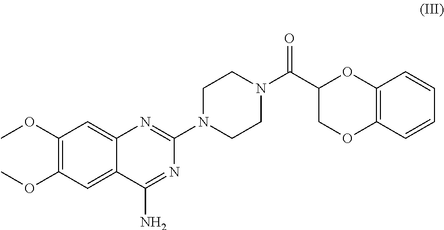 [SMILES: COc1cc2nc(N3CCN(C(=O)C4COc5ccccc5O4)CC3)nc(N)c2cc1OC]

or a pharmaceutically acceptable salt or solvate thereof; for use in the treatment or prophylaxis of Attention Deficit/Hyperactivity Disorder (ADHD).

The present invention further provides a method for the treatment and/or prophylaxis of ADHD, comprising a step of administering a dose of the compound of formula (I), (II), (S-AM), or (III) to a patient known to have, suspected of having, or at risk of developing ADHD.

The present inventors have found that in a zebrafish (Danio rerio) model of ADHD, the compounds of formula (I), (II), (S-AM), and (III) were surprisingly effective at reducing ADHD symptoms such as hyperactivity and motor impulsivity in Lphn3.1 knock-out zebrafish larvae.

The invention also provides the use of the compound according to formula (I), (II), (S-AM), or (III) for the manufacture of a medicament for the treatment and/or prophylaxis of ADHD.

The present invention further provides a kit comprising the compound according to formula (I), (II), (S-AM), or (III); and one or more further pharmacological interventions, instructions for a dietetic intervention and/or instructions for a psychological intervention. The kit of the present invention finds use in the treatment or prophylaxis of ADHD.

### DESCRIPTION OF THE DRAWINGS

FIG. 1 shows a spectrogram of a 24 hour recording of the velocity (mm/s) of zebrafish larvae with a homozygous knock-out of the Lphn3.1 gene (herein referred to as “Lphn3.1 HOM”) and zebrafish larvae with a wild-type Lphn3.1 gene (herein referred to as “Lphn3.1 WT”). Both groups of larvae were administered a vehicle control (i.e. DMSO only). The grey area shown on the spectrogram indicates the lights-off phases of the experiment (five 30 minute phases, the first phases starting at 1.30 pm and followed by a 10 hour night period wherein lights were off, starting at 10:00 p.m. and ending at 8:00 a.m. the morning after).

FIG. 2 shows a bar graph that depicts the differences in average distance moved by Lphn3.1 HOM and Lphn3.1 WT larvae during the five 30 minute lights-on phases. Non-significant interaction was observed between the genotype and vehicle (i.e. DMSO) (f=1.003, df=1, p=0.317). A significant effect of genotype on distance moved, between Lphn3.1 HOM and Lphn3.1 WT larvae, was observed (f=117.67, df=1, p<0.001). A non-significant effect of the vehicle was demonstrated between naive (i.e. larvae that received neither a test compound or a vehicle control) and DMSO treated larvae (f=0.222 df=1, p=0.638).

FIG. 3 shows a comparison of the distance moved by Lphn3.1 HOM larvae that received aceclofenac, atomoxetine (referred to as tomoxetin hydrochloride in FIG. 3), or a vehicle control (i.e. DMSO only). The dashed line on the plot indicates the average distance moved by Lphn3.1 HOM larvae over five lights-on phases following treatment with the vehicle control (n=189). The dotted line indicates the average distance moved by Lphn3.1 HOM larvae over the five lights-on phases following treatment with 1 μM atomoxetine hydrochloride (n=24). The dots indicate the effect of aceclofenac at a dosage of 1 μM, 10 μM or 30 μM on the average distance moved by Lphn3.1 HOM larvae over the five lights-on phases (n=24) (all data presented with +/−SEM). Statistically significant differences were found between larvae treated with aceclofenac and larvae that received the vehicle control (f=3.479, df=3, p=0.017, second round: f=7.035, df=3, p<0.001).

FIG. 4 shows a comparison of the distance moved by Lphn3.1 HOM larvae that received amlodipine, atomoxetine (referred to as tomoxetin hydrochloride in FIG. 4), or a vehicle control (i.e. DMSO only). The dashed line on the plot indicates the average distance moved by Lphn3.1 HOM larvae over five lights-on phases following treatment with the vehicle control (n=189). The dotted line indicates the average distance moved by Lphn3.1 HOM larvae over the five lights-on phases following treatment with 1 μM atomoxetine hydrochloride (n=24). The dots indicate the effect of amlodipine at a dosage of 1 μM, 10 μM or 30 μM on the average distance moved by Lphn3.1 HOM larvae over the five lights-on phases (n=24) (all data presented with +/−SEM). Statistically significant differences were found between larvae treated with amlodipine and larvae that received the vehicle control (f=23.684, df=3, p<0.001, second round: f=31.699, df=3, p<0.001).

FIG. 5 shows a comparison of the distance moved by Lphn3.1 HOM larvae that received doxazosin, atomoxetine (referred to as tomoxetin hydrochloride in FIG. 5), or a vehicle control (i.e. DMSO only). The dashed line on the plot indicates the average distance moved by Lphn3.1 HOM larvae over five lights-on phases following treatment with the vehicle control (n=189). The dotted line indicates the average distance moved by Lphn3.1 HOM larvae over the five lights-on phases following treatment with 1 μM atomoxetine hydrochloride (n=24). The dots indicate the effect of doxazosin at a dosage of 1 μM, 10 μM or 30 μM on the average distance moved by Lphn3.1 HOM larvae over the five lights-on phases (n=24) (all data presented with +/−SEM). Statistically significant differences were found between larvae treated with doxazosin and larvae that received the vehicle control (f=14.642, df=3, p<0.001, second round: f=29.29, df=3, p<0.001).

FIG. 6 Amlodipine crosses the blood-brain barrier in adult adgrl3.1−/− zebrafish after a 30-min 10 μM exposure.

FIG. 7 Plasma and brain amlodipine concentrations 6 h after single- or repeated-dose oral administration (1 or 10 mg/kg) in Sprague-Dawley rats.

FIG. 8 K_{p,uu} values 6 h after single- or repeated-dose oral administration (1 or 10 mg/kg) in Sprague-Dawley rats.

FIG. 9 Sex-stratified plasma and brain amlodipine concentrations in CRH mice after repeated intraperitoneal dosing with amlodipine (10 mg/kg/day×5).

FIG. 10 K_{p,uu} values in CRH mice after repeated intraperitoneal dosing with amlodipine (10 mg/kg/day×5).

FIG. 11 Average distance moved during lights-on phases for all treatment groups.

FIG. 12 Brain transcriptomics after S-amlodipine exposure in adult zebrafish identifies 3 differentially expressed genes (3 upregulated) at fold-change ≥1.2 and FDR ≤0.05.

FIG. 13 Brain transcriptomics after R-amlodipine exposure in adult zebrafish identifies 22 differentially expressed genes (14 upregulated; 8 downregulated) at fold-change ≥1.2 and FDR ≤0.05.

FIG. 14 Brain proteomics after S-amlodipine exposure in adult zebrafish identifies 78 differentially expressed proteins (33 upregulated; 45 downregulated) at fold-change ≥1.5 and p-value ≤0.05.

FIG. 15 Brain proteomics after R-amlodipine exposure in adult zebrafish identifies 87 differentially expressed proteins (44 upregulated; 43 downregulated) at fold-change ≥1.5 and p-value ≤0.05.

FIG. 16 Brain metabolomics after S-amlodipine exposure in adult zebrafish identifies 199 differentially expressed metabolites (86 upregulated; 113 downregulated) at fold-change ≥1.2 and p-value ≤0.05.

FIG. 17 Brain metabolomics after R-amlodipine exposure in adult zebrafish identifies 187 differentially expressed metabolites (94 upregulated; 93 downregulated) at fold-change ≥1.2 and p-value ≤0.05.

### DETAILED DESCRIPTION

The inventors of the present invention have found that the compounds according to formula (I), (II), (S-AM), and (III) are surprisingly effective for the treatment or prophylaxis of ADHD.

As discussed in more detail below, the present inventors have found that aceclofenac, amlodipine, the S-enantiomer of amlodipine (S-AM), and doxazosin are surprisingly effective at reducing the activity of zebrafish larvae in a model of ADHD. The ADHD model used by the present inventors uses zebrafish carrying a homozygous knock-out of the Latrophilin 3 (Lphn3.1) gene. Mutations in the corresponding Lphn.3.1 gene in humans (i.e. LPHN3) have been strongly implicated as a risk factor for ADHD in humans (Arcos-Burgos et al, 2010, Mol Psychiatry 15, 1053-1066, and Lange et al., Prog Neuropsychopharmacol Biol Psychiatry, 2018, 84(A), 181-189). The function of the Lphn.3.1 gene in zebrafish has been found to correlate with the function observed in humans. In the zebrafish model used by the present inventors, ADHD-like behavioural phenotypes, such as hyperactivity and motor impulsivity, are observed in zebrafish larvae carrying a homozygous knock-out of the Lphn3.1 gene. Using the zebrafish model of ADHD, the present inventors have identified aceclofenac, a non-steriodal anti-inflammatory drug (NSAID) typically used to treat osteoarthritis, rheumatoid arthritis and ankylosing spondylitis, as being surprisingly effective at reducing ADHD behaviours such as hyperactivity and motor impulsivity. The present inventors have also identified, using the zebrafish model of ADHD, amlodipine, particularly the S-enantiomer of amplodipine (S-AM), a dihydropyridine calcium channel blocker typically used to treat high blood pressure and angina, and doxazosin, an alpha-1 antagonist typically used to treat hypertension and benign prostatic hypertrophy symptoms, as being surprisingly effective at reducing ADHD behaviours such as hyper-activity and motor impulsivity.

Thus, the present invention provides the compound of formula (I), (II), (S-AM), or (III) (i.e. aceclofenac, amlodipine, S-amlodipine, or doxazosin, respectively), or a pharmaceutically acceptable salt or solvate thereof, for use in the treatment or prophylaxis of ADHD.

For the avoidance of doubt, in this document, it is intended that the compound of formula (I), (II), (S-AM), or (III) includes all salts and solvates thereof, unless stated otherwise.

The compound of formula (I), (II), (S-AM), or (III) may be prepared using methods known to those skilled in the art of organic chemistry. Pharmaceutical preparations of the compound of formula (I), (II), (S-AM), or (III) suitable for use in the present invention are available from commercial sources. For example, pharmaceutical preparations of the compound of formula (I) are marketed under the name PRESERVEX®, pharmaceutical preparations of the compound of formula (II) are marketed under the name ISTIN®, and pharmaceutical preparations of the compound of formula (III) are marketed under the name CARDURA®. Generic pharmaceutical preparations of the compound of formula (I), (II), or (III) are also available.

For the avoidance of doubt, in this document, it is intended that the compounds of formula (I), (II), (S-AM), and (III) include all enantiomers thereof, unless stated otherwise. Also for the avoidance of doubt, in this document, it is intended that the compounds of formula (I), (II), (S-AM), and (III) include compounds that differ by immaterial structural variations, and therefore expected to display similar properties to aceclofenac, amlodipine, S-AM, and doxazosin in the ADHD model described herein.

The compound of formula (I), (II), (S-AM), or (III) may form salts or solvates. Salts of the compound of formula (I), (II), (S-AM), or (III) which are suitable for use in the present invention are those wherein a counterion is pharmaceutically acceptable. However, the use of salts having non-pharmaceutically acceptable counter-ions are within the scope of the present invention, for example, for use as intermediates in the preparation of the compound of formula (I), (II), (S-AM), or (III) and their pharmaceutically acceptable salts, and physiologically functional derivatives. Suitable salts for use according to the invention include those formed with organic or inorganic acids. In particular, suitable salts formed with acids for use according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxy-carboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxalic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Suitable cations which may be present in salts include alkali metal cations, especially sodium, potassium and calcium, and ammonium or amino cations.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2nd ed R. Lui CRC Press, page 553 and Byrn et al Pharm Res 12(7), 1995, 945-954. Before it is made up in solution, the compound of formula (I), (II), (S-AM), or (III) may be in the form of a solvate. Solvates of the compound of formula (I), (II), (S-AM), or (III) which are suitable for use as a medicament according to the invention are those wherein the associated solvent is pharmaceutically acceptable. For example, a hydrate is a pharmaceutically acceptable solvate.

A compound which, upon administration to the recipient, is capable of being converted into the compound of formula (I), (II), (S-AM), or (III), or an active metabolite or residue thereof, is known as a “prodrug”. Thus, in certain embodiments, the compound of formula (I), (II), (S-AM), or (III) may be provided in the form of a prodrug. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

### Pharmaceutical Compositions

While it is possible for the compound of formula (I), (II), (S-AM), or (III) to be administered alone, it is preferable for it to be present in a composition and particularly in a pharmaceutical composition. Pharmaceutical compositions of the present invention comprise the compound of formula (I), (II), (S-AM), or (III); and one or more pharmaceutically acceptable excipients.

Pharmaceutical compositions include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration, although the most suitable route may depend upon the characteristics of the subject under treatment, for example the species, age, weight, sex, medical conditions, the particular type of ADHD (for example, “impulsive type/hyperactive type”, “inattentive type” and “combined type” ADHD) and its severity, and other relevant medical and physical factors.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind described herein. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Pharmaceutical compositions suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The compound of formula (I), (II), (S-AM), or (III) may also be presented as a bolus, electuary or paste. The pharmaceutical compositions may optionally be present in a form that provides slow or controlled release of the compound of formula (I), (II), (S-AM), or (III) once administered to a subject. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Preferred unit dosage compositions are those containing an exploratory dose or therapeutic dose, or an appropriate fraction thereof, of the compound of formula (I), (II), (S-AM), or (III).

In preferred embodiments, a composition for use according to the present invention consists essentially of the compound of formula (I), (II), (S-AM), or (III); and at least one pharmaceutically acceptable excipient.

An exemplary composition comprising the compound of formula (I) for use according to the present invention comprises one or more of the following pharmaceutically acceptable carriers: microcrystalline cellulose, sodium croscarmellose, copovidone, glyceryl palmitostearate, polyvinylpyrrolidone (for example, povidone K30), hypromellose, polyethylene glycol, anhydrous colloidal silica, glycerol distearate, talc, titanium dioxide. A further exemplary composition comprising the compound of formula (I) for use according to the present invention is a tablet with a core and a coating, wherein the core comprises microcrystalline cellulose, sodium croscarmellose, povidone and glyceryl palmitostearate, and the coating comprises hypromellose, polyethylene glycol, and titanium dioxide.

An exemplary composition comprising the compound of formula (II) or (S-AM) for use according to the present invention comprises one or more of the following pharmaceutically acceptable carriers: calcium hydrogen phosphate, microcrystalline cellulose, sodium starch glycolate, magnesium stearate, sodium acid citrate, sodium croscarmellose, crospovidone, anhydrous calcium hydrogen phosphate, anhydrous colloidal silica. A further exemplary composition comprising the compound of formula (II) or (S-AM) for use according to the present invention is a tablet comprising: sodium starch glycolate, anhydrous calcium hydrogen phosphate, microcrystalline cellulose, magnesium stearate. A further exemplary composition comprising the compound of formula (II) or (S-AM) for use according to the present invention is an oral solution comprising: methyl parahydroxybenzoate, propylene glycol, anhydrous disodium phosphate, sodium dihydrogen phosphate dihydrate, purified water, glycerol, maltitol liquid.

An exemplary composition comprising the compound of formula (III) for use according to the present invention comprises one or more of the following pharmaceutically acceptable carriers: lactose, magnesium stearate, polyvinylpyrrolidone (for example, povidone K29-32), sodium stearyl fumarate, microcrystalline cellulose, sodium lauryl sulphate, sodium starch glycolate, butylhydroxytoluene, tocopherol, anhydrous colloidal silica, polyethylene oxide, sodium chloride, hypromellose, iron oxide, titanium dioxide, cellulose acetate, Macrogol, ammonium hydroxide, propylene glycol, methacrylic acid/ethyl acetate copolymer. A further exemplary composition comprising the compound of formula (III) for use according to the present invention is a tablet with a core and a coating, wherein the core comprises polyethylene oxide, microcrystalline cellulose, povidone, butylhydroxytoluene, a-tocopherol, colloidal anhydrous silica, and sodium stearyl fumarate, and the coating comprises methacrylic acid/ethyl acrylate copolymer, colloidal anhydrous silica, Macrogol, and titanium dioxide.

It should be understood that in addition to the ingredients particularly mentioned above, the compositions for use in this invention may include other agents conventional in the art having regard to the type of composition in question.

The compositions for use according to the invention may comprise one or more further therapeutic agents. Examples of further therapeutic agents that may be present in a composition for use according to the present invention include, but are not limited to, methylphenidate, amphetamine (for example dexamphetamine), lisdexamfetamine, atomoxetine, viloxazine, clonidine, and guanfacine. Typically, one or more further therapeutic agents is a stimulant, such as amphetamine, methylphenidate and lisdexamfetamine.

### Attention Deficit/Hyperactivity Disorder (ADHD)

The present inventors have found that the compounds of formula (I), (II), (S-AM), and (III), and pharmaceutical compositions thereof, are particularly effective at reducing the symptoms of ADHD in a subject known or suspected of having ADHD, or delaying the onset of, or preventing ADHD in a subject known or suspected of being at risk of developing ADHD.

Thus, the compound of formula (I), (II), (S-AM), or (III), or a pharmaceutical composition thereof, for use according to the present invention, may be administered to a subject known or suspected of having ADHD, or known or suspected of being at risk of developing ADHD. The subject may be a human subject, for example a human patient. The human subject may be a child (e.g. under the age of about 10 years old), an adolescent (e.g. between the ages of about 10 to 19 years old) or may be an adult (e.g. over the age of about 18 to 21 years old).

The subject known or suspected of having ADHD may have ADHD that is characterised by impulsive and hyperactive behaviours without impaired levels of attention (i.e. “impulsive type/hyperactive type” ADHD). Or, the subject known or suspected of having ADHD may have ADHD that is characterised by impaired levels of attention without hyperactivity or impulsivity (i.e. “inattentive type” ADHD). Or, the subject known or suspected of having ADHD may have ADHD that is characterised by a combination of reduced levels of attention with hyperactivity and impulsivity behaviours (i.e. “combined type” ADHD).

A subject known or suspected of being at risk of developing ADHD may be one who has a known or suspected genetic predisposition for developing ADHD, for example, the subject may have a mutation in the Latrophilin 3 (LPHN3) gene. Examples of further genetic mutations implicated in ADHD have been reported (see, for example, Faraone and Larsson, Molecular Psychiatry, 2019, 24, 562-575, which is incorporated herein by reference), and include, for example, mutations in one or more of the following genes: the serotonin transporter (5HTT) gene, the dopamine transporter (DAT1) gene, the D4 dopamine receptor (DRD4) gene, the D5 dopamine receptor (DRD5) gene, the serotonin 1B receptor gene (HTR1B) and the Synaptosomal-Associated Protein (SNAP25) gene. Additionally, or alternatively, a subject known or suspected of being at risk of developing ADHD may be one who has been exposed to one or more potential environmental risk factors associated with ADHD, such as maternal pre-pregnancy obesity, pre-eclampsia, hypertension, acetaminophen exposure, and smoking during pregnancy; and childhood atopic diseases.

Additionally, the subject known or suspected of having ADHD, or known or suspected of being at risk of developing ADHD, may also have signs or symptoms of other conditions such as depression, anxiety disorder, oppositional defiant disorder (ODD), conduct disorder, sleeps problems (for example, sleep-onset insomnia), autism spectrum disorder (ASD), bipolar disorder, epilepsy, Tourette's syndrome and/or leaning difficulties such as dyslexia. Typically, the subject is one who meets the diagnostic criteria for ADHD set out in the Diagnostic and Statistical Manual of Mental Disorders (DSM) or International Classification of Diseases (ICD).

Additionally, the subject known or suspected of having ADHD, or known or suspected of being at risk of developing ADHD, may also suffer from a substance abuse disorder, for example addiction and/or abuse of stimulants such as amphetamine.

For example, the compound of formula (I), (II), (S-AM), or (III), or compositions thereof, for use according to the invention, may be administered to a subject for whom one or more established stimulant-based ADHD treatments (for example, methylphenidate, dexamphetamine, and lisdexamfetamine) are deemed to be unsuitable due to the subject being at risk, having a history of, or currently suffering from, a substance abuse disorder.

The present inventors have shown using a zebrafish model of ADHD that the compound of formula (I), (II), (S-AM), or (III) is surprisingly effective at reducing hyperactivity and motor impulsivity. The efficacy of the compound of formula (I), (II), (S-AM), or (III) at reducing hyperactivity and motor impulsivity in the zebrafish model of ADHD makes the compound of formula (I), (II), (S-AM), or (III) an especially attractive treatment for ADHD that is characterised by hyperactivity and/or impulsivity behaviours (i.e. “impulsive type/hyperactive type” ADHD or “combined type” ADHD). Thus, in certain embodiments, the compound of formula (I), (II), (S-AM), or (III) or a pharmaceutical composition thereof, for use according to the present invention, may be administered to a subject known or suspected of having ADHD that is characterised by impulsive and hyperactive behaviours without impaired levels of attention (i.e. “impulsive type/hyperactive type” ADHD), or ADHD that is characterised by a combination of reduced levels of attention with hyperactivity and impulsivity behaviours (i.e. “combined type” ADHD).

The compound of formula (I), (II), (S-AM), or (III), or compositions thereof, for use according to the invention, may be administered to a subject for whom one or more established ADHD treatments (for example, methylphenidate, dexamphetamine, lisdexamfetamine, atomoxetine, viloxazine, guanfacine and clonidine) have been ineffective at reducing one or more of the symptoms of ADHD and/or induced intolerable adverse effects. Examples of adverse effects known or suspected of being caused by established ADHD treatments include impaired sleep, lack of appetite, increased blood pressure, stunted growth, disruption of circadian rhythm and neurotoxicity.

The compound of formula (I), (II), (S-AM), or (III), and compositions thereof, as described herein finds utility in a method of treating or preventing ADHD, wherein said method comprises a step of administering the compound of formula (I), (II), (S-AM), or (III), or a composition thereof, to a patient known or suspected of having ADHD, or known or suspected of being at risk of developing ADHD. The compound of formula (I), (II), (S-AM), or (III) also finds use in the manufacture of a medicament for the treatment or prophylaxis of ADHD.

### Dosage Regimens

The amount of the compound of formula (I), (II), (S-AM), or (III) which is required to achieve a therapeutic effect will vary with the particular route of administration and the characteristics of the subject under treatment, for example the species, age, weight, sex, medical conditions, the particular type of ADHD (for example “impulsive type/hyper-active type”, “inattentive type” and “combined type” ADHD) and its severity, and other relevant medical and physical factors. An ordinarily skilled physician can readily determine and administer an effective amount of the compound of formula (I), (II), (S-AM), or (III) required for treatment or prophylaxis of ADHD.

The compound of formula (I), (II), (S-AM), or (III) may be administered daily (including several times daily), every second or third day, weekly, every second, third, or fourth week or even as a single dose depending on the subject and the characteristics of the ADHD to be treated. The compound of formula (I), (II), (S-AM), or (III) may further be administered as a depot formulation, the dosage and frequency of administration depending upon the on the subject and the characteristics of the ADHD to be treated.

The compound of formula (I) (excluding the mass of any counterion or solvent) may be administered in an amount of about 1 mg to 1000 mg per administration. For example, at least 1 mg, at least 5 mg, at least 10 mg, at least 15 mg, at least 20 mg, at least 25 mg, at least 40 mg, at least 50 mg, at least 60 mg, at least 70 mg, at least 80 mg, at least 90 mg, at least 100 mg, at least 150 mg, at least 200 mg, at least 300 mg, at least 400 mg, at least 500 mg, at least 600 mg, at least 700 mg, at least 800 mg, at least 900 mg or 1000 mg may be administered to a subject.

Typically, the compound of formula (I) is administered as a single daily dose of about 100 mg, 200 mg, 300 mg, 400 mg, 500 mg or 600 mg (excluding the mass of any counterion or solvent). Alternatively, the compound of formula (I) is administered as a dose of 100 mg, 150 mg, 200 mg, 250 mg or 300 mg (excluding the mass of any counterion or solvent), two, three or four times a day. For example, a daily dose of 100 mg per day may be administered as two separate doses of 50 mg, wherein the first dose is administered in the morning and the second dose is administered in the evening (for example, after 6 p.m.). Or, for example, a daily dose of 200 mg per day may be administered as two separate doses of 100 mg, wherein the first dose is administered in the morning and the second dose is administered in the evening.

The compound of formula (II) (excluding the mass of any counterion or solvent) may be administered in an amount of about 0.1 mg to 10 mg per administration. For example, at least 0.1 mg, at least 0.5 mg, at least 1 mg, at least 5 mg, at least 2 mg, at least 2.5 mg, at least 3 mg, at least 4 mg, at least 5 mg, at least 6 mg, at least 7 mg, at least 8 mg, at least 9 mg, at least 10 mg may be administered to a subject.

Typically, the compound of formula (II) is administered as a single daily dose of about 1 mg, 1.5 mg, 2 mg, 2.5 mg, 3 mg, 3.5 mg, 4 mg, 4.5 mg, 5 mg or 10 mg (excluding the mass of any counterion or solvent). For example, a single daily dose of 2 mg, 2.5 mg, 5 mg, 7.5 mg or 10 mg. Alternatively, the compound of formula (II) is administered as a dose of about 1 mg, 1.5 mg, 2 mg, 2.5 mg, 3 mg, 3.5 mg, 4 mg, 4.5 mg or 5 mg (excluding the mass of any counterion or solvent), two, three or four times a day. For example, a daily dose of 5 mg per day may be administered as two separate doses of 2.5 mg, wherein, for example, the first dose is administered in the morning and the second dose is administered in the evening (for example, after 6 p.m.). Or, for example, a daily dose of 10 mg per day may be administered as two separate doses, wherein, for example, a first dose of 5 mg is administered in the morning and the second dose of 5 mg is administered in the evening.

The compound of formula S-AM (excluding the mass of any counterion or solvent) may be administered in an amount of about 0.1 mg to 5 mg per administration. For example, at least 0.1 mg, at least 0.5 mg, at least 1 mg, at least 5 mg, at least 2 mg, at least 2.5 mg, at least 3 mg, at least 4 mg, at least 5 mg may be administered to a subject.

Typically, the compound of formula S-AM is administered as a single daily dose of about 0.1 mg, 1 mg, 1 mg, 2 mg, 2.5 mg, 3 mg, 3.5 mg, 4 mg, 4.5 mg, 5 mg (excluding the mass of any counterion or solvent). For example, a single daily dose of 0.1 mg, 1 mg, 2 mg, 2.5 mg or 5 mg. Alternatively, the compound of formula S-AM is administered as a dose of about 0.1 mg, 1 mg, 1.5 mg, 2 mg, 2.5 mg, 3 mg, 3.5 mg, 4 mg, 4.5 mg or 5 mg (excluding the mass of any counterion or solvent), two, three or four times a day. For example, a daily dose of 5 mg per day may be administered as two separate doses of 2.5 mg, wherein, for example, the first dose is administered in the morning and the second dose is administered in the evening (for example, after 6 p.m.).

The compound of formula (III) (excluding the mass of any counterion or solvent) may be administered in an amount of about 0.1 mg to 16 mg per administration. For example, at least 0.5 mg, at least 1 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg may be administered to a subject. Typically, the compound of formula (III) is administered as a single daily dose of 1 mg, 2 mg, 4 mg, 8 mg or 16 mg (excluding the mass of any counterion or solvent). Alternatively, the compound of formula (III) is administered as a dose of 1 mg, 2 mg, 4 mg, 8 mg or 16 mg (excluding the mass of any counterion or solvent), two, three or four times a day.

In certain embodiments, the compound of formula (I), (II), (S-AM), or (III) is administered as a composition. Preferably, the composition is a pharmaceutical composition for use according to the present invention.

Whilst the compound of formula (I), (II), (S-AM), or (III) may be used as the sole active ingredient in the present invention, it is also possible for it to be used in combination with one or more further therapeutic interventions, and the use of such combinations provides one embodiment of the present invention. Examples of further therapeutic interventions include pharmacological interventions, dietetic interventions, and psychological intervention.

Further pharmacological interventions may be therapeutic agents useful in the treatment or prophylaxis of ADHD, or other pharmaceutically active materials. Such agents are known in the art. Examples of further therapeutic agents for use in the present invention include those described herein. Typically, the further therapeutic agent is a stimulant, such as amphetamine, methylphenidate, and lisdexamfetamine. Examples of suitable dietetic interventions include, for example, supplementary fatty acids and the exclusion of artificial food colour from the diet. Examples of suitable psychological interventions include, for example, cognitive behavioural therapy (CBT).

The one or more further therapeutic interventions may be used simultaneously, sequentially or separately with/from the administration of the compound of formula (I), (II), (S-AM), or (III). The individual components of such combinations can be administered separately at different times during the course of therapy or concurrently in divided or single combination forms. An ordinarily skilled physician can readily determine and administer the effective amount of one or more therapeutic interventions required to have the desired therapeutic effect.

Preferred unit dosage compositions for use according to the invention are those containing an effective dose, or an appropriate fraction thereof, of the compound of formula (I), (II), (S-AM), or (III). The release of the compound of formula (I), (II) or (III) from certain composition may also be sustained, for example, if the composition contains suitable controlled-release excipients.

### Kits

The present invention provides a kit comprising the compound of formula (I), (II), (S-AM), or (III), one or more pharmaceutically acceptable excipients, and optionally one or more further therapeutic agents that are useful in the treatment or prophylaxis of ADHD. Examples of such further therapeutic agents include those described herein as being suitable for use in the present invention, and being optionally present in a pharmaceutical composition of the invention as a further therapeutic agent.

Kits of the present inventions may also contain instructions for a dietetic intervention and/or instructions for a psychological intervention suitable for ADHD. For example, the kits may include instructions for a nutrition plan suitable for the subject receiving treatment and/or the kit may comprise instructions/guidance for cognitive behavioral therapy (CBT).

Kits of the present invention find use in the treatment and prophylaxis of ADHD.

For the avoidance of doubt, the compound of formula (I), (II), (S-AM), or (III) present in a kit according to the present invention is in a form and quantity suitable for use according to the present invention. Suitable pharmaceutical compositions and formulations are described herein. The skilled person can readily determine a quantity of the compound of formula (I), (II), (S-AM), or (III) suitable for including in a kit of the invention, and for use according to the invention.

### Metabolites

Active metabolites of the compound of formula (I), (II), (S-AM), or (III) may be used in the treatment or prophylaxis of Attention Deficit/Hyperactivity Disorder (ADHD). In embodiments wherein the compound is an active metabolite of a compound of formula (I), (II), (S-AM), or (III), the compound may be isolated from cells treated with the compound of formula (I), (II), (S-AM), or (III), or may be prepared using standard organic chemistry techniques. For the avoidance of doubt, when the compound for use according to the present invention is an active metabolite of a compound of formula (I), (II), (S-AM), or (III), the compound may be used in the form of a composition and/or as medicaments in the same manner as described herein for the compound of formula (I), (II), (S-AM), or (III).

In certain embodiments, the compound for use according to the present invention is an active metabolite of the compound of formula (I). The compound of formula (I) is known to be metabolised in humans into the active metabolites, 4-hydroxy-aceclofenac, diclofenac and 4-hydroxy-diclofenac. Thus, in certain embodiments, the present invention also provides a metabolite of the compound of formula (I) for use in the treatment or prophylaxis of Attention Deficit/Hyperactivity Disorder (ADHD), wherein the metabolite is a compound according to formula (IV):

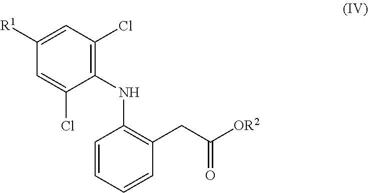

Wherein, when R¹ is hydrogen or OH; and when R¹ is hydrogen, then R² is hydrogen, and when R¹ is OH, then R² is hydrogen or —CH2COOH; or a pharmaceutically acceptable salt or solvate thereof.

In certain embodiments, in the compound of formula (IV), R¹ and R² are each hydrogen. That is to say that the compound of formula (IV) is diclofenac. When the compound of formula (IV) is diclofenac, the compound may be obtained from commercial sources. For example, when the compound of formula (IV) is diclofenac, the compound may be the product marketed under the name VOLTAREN®, or a generic pharmaceutical preparation thereof.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

### A Compound According to Formula (I), (II), or (III)

Danio rerio is gaining popularity in biological psychiatry. Their rich behavioral repertoire, the availability of well-established behavioral and automated behavioral assays, make zebrafish a useful model of various human brain disorders. The neuronal pathways involved in brain physiology are highly conserved, including all major neurotransmitter systems and high genetic homology. Gene editing technology allows precise modelling of human disorders. The Lphn3.1 knock-out model of ADHD described herein robustly exhibits the hallmarks of human ADHD, that is: hyperactivity, hypersensitivity to known dopamine agonists, and rescue of the phenotype following administration of known anti ADHD compounds.

### Material and Methods

Zebrafish larvae carrying a knock-out of the Latrophilin3 (Lphn3.1) gene were generated by CRISPR-Cas9. Zebrafish were kept in a 14:10 light:dark cycle in 3 or 10 L multi tank constant flow system (Aquatic Habitats, Apopka, FL, USA). For behavioral analysis a total number of 881, 6 days-post fertilization (dpf) embryos carrying a homozygous knock-out of the Lphn3.1 gene were used in the study. Adult zebrafish with a homozygous knock-out of the Lphn3.1 gene and adult zebrafish with a wild-type Lphn3.1 gene were fed three times a day on a variable diet of TetraMin flakes (Tetra Holding GmbH, Melle, Germany) and live Artemia. Water temperature was held at a constant 28.5° C. and replaced at a rate of 10% per day. Eggs were collected between 10:00-12:00 a.m. and contained in 2 L tanks. The following day, dead eggs were removed and tanks were cleaned. Eggs were incubated for 4 days at 28.5° C. in system water mixed with methylene blue. All procedures in the study were carried out in strict compliance with the regulations of, and approved by, the National Bioethics Committee of Iceland (regulation 460/2017).

### Genotyping

Strains were verified for knock-down of the Lphn3.1 gene using Western blot.

### Behavioral Recordings

At 5 dpf, larvae were placed in individual wells of 96-microwell plates (Nunc, Roskilde, Denmark) in system water. The microwell plates were relocated to a custom-built activity monitoring system fitted with 24 infrared cameras (Ikegami, ICD-49E; Ikegami Tsushinki Co, Japan) which was thermo-regulated at 28.5° C., blocked from daylight and illuminated from below with white (255 lx; light-phase) and infrared light (0 lx; dark-phase). Larvae behavior was tracked in two dimensions at 5 Hz. Larvae were left to acclimatize in the activity monitoring system for 24 hours prior to recording. Exclusion criterion was based on the percentage of samples during a recording where a larva was not tracked. The threshold was set at 10%, thus a larva that was tracked <90% of the total recording time was excluded from the study.

### Motor Assay

Following a 24 hour acclimation period, behavioral recordings were made. Locomotor activity was recorded between 1.00 pm and 6.00 pm at 6 dpf during alternating light and dark conditions, presented in 30-minute intervals. For this period, average distance moved (mm) by the larvae was calculated as the mean of the total distance swum during five separate 30-minute intervals immediately after transition of light conditions.

### Example 1: Efficacy of Aceclofenac, Amlodipine or Doxazosin Compared to Atomoxetine in the Zebrafish Model of ADHD

Zebrafish larvae with a homozygous knock-out of the Lphn3.1 gene (herein referred to as “Lphn3.1 HOM”) and zebrafish larvae with a wild-type Lphn3.1 gene (herein referred to as “Lphn3.1 WT”) were exposed to the on-the-market ADHD drug atomoxetine hydrochloride (tomoxetine hydrochloride), aceclofenac (Prestwick, 67400 Illkirch, France), amlodipine (Prestwick, 67400 Illkirch, France), doxazosin (Prestwick, 67400 Illkirch, France), or a vehicle control before behavioral recordings started.

Drug preparation was performed on the day of recording. Drugs were diluted from stock solution using distilled water (Invitrogen, Paisley, PA4 9RF, UK). Three different concentration of each drug were used, 1 μM, 10 μM and 30 μM. Additionally, 0.03% DMSO (Sigma-Aldrich, St. Louis, USA) solution was prepared for the vehicle control group. Drugs and vehicle control were added into the microwells on the day of recording, between 11.30 a.m. and 12.30 p.m.

Data was obtained using EthoVision XT (Version 11.5.2016, Noldus) and exported to Microsoft Excel for analysis. Statistical analysis was performed using IBM SPSS Statistics for Windows, version 26 (IBM corp., Armonk, N.Y., USA). Figures were produced using Microsoft Excel and GraphPad Prism Software (Version 5.01, GraphPad Software Inc.). Data are presented as mean±standard error of the mean (s.e.m). For analysis of Lphn 3.1 naïve (i.e. untreated larvae that do not receive a test compound or a vehicle control) and DMSO larvae (i.e. larvae receiving only the DMSO vehicle control), statistical differences were evaluated using two-way ANOVA. For analysis of Lphn 3.1 larvae treated with aceclofenac statistical differences were evaluated using one-way ANOVA with Dunnett two-sided post hoc analysis. P<0.05 was considered statistically significant.

### Results

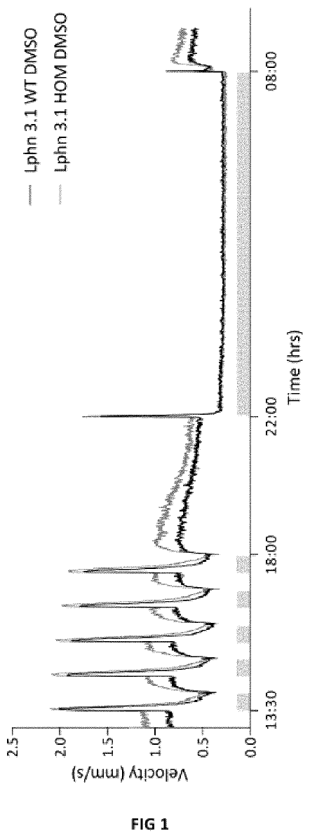

Following 24 hours of recording it was evident that the Lphn3.1 HOM larvae display a notable hyperactive phenotype. Higher peak velocities following lights-off (which results in a stereotypical short, increase in activity for zebrafish), as well as higher overall average velocity during the lights-on periods, were observed for Lphn3.1 HOM larvae (FIG. 1). In FIG. 1, the activity of the Lphn3.1 HOM larvae is shown in the upper line and the activity of the Lphn3.1 WT larvae is shown in the lower line. The overall activity pattern of the larvae suggested that any period could be selected for statistical analysis.

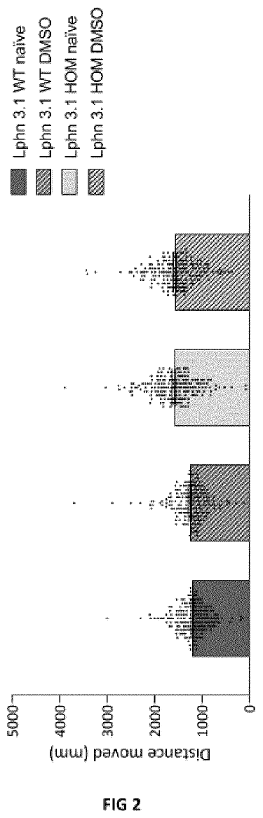

Using the average distance moved during lights-on periods it was demonstrated, first, that homozygous larvae were indeed statistically significantly hypermotile compared to wild-types and, second, the vehicle (DMSO) had no effects on behavior (FIG. 2).

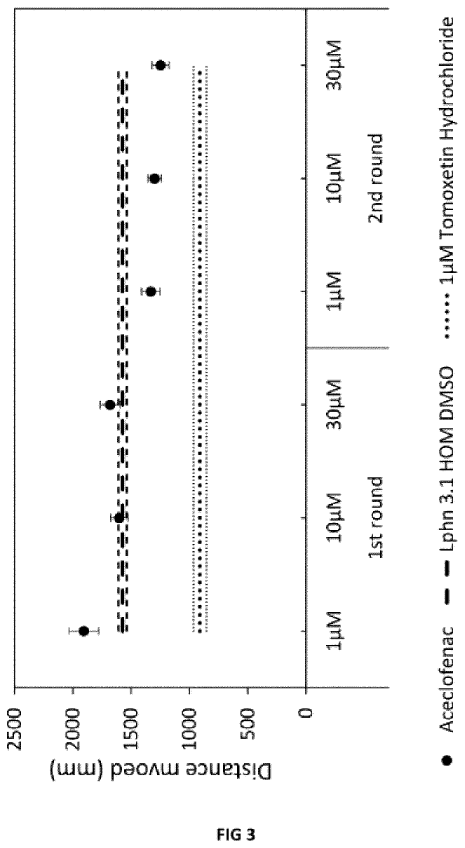

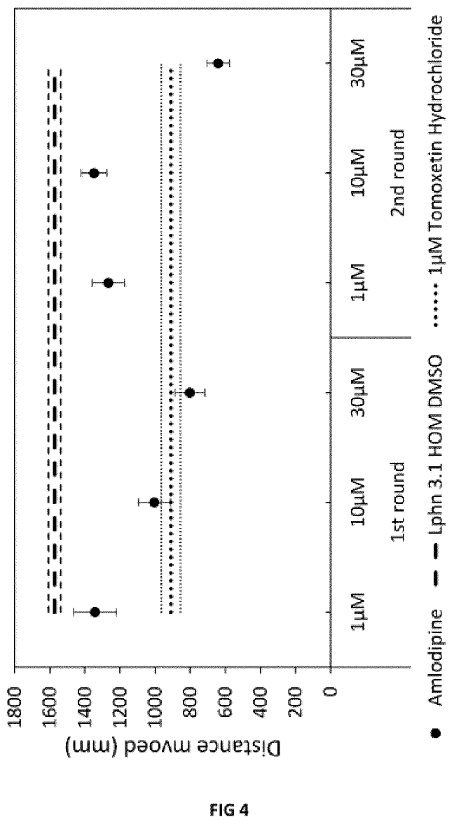

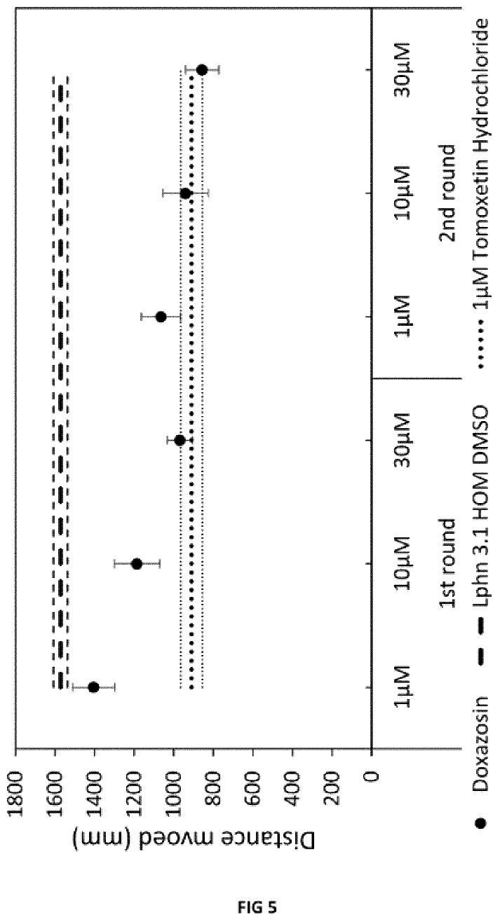

Atomoxetine, aceclofenac, amlodipine, and doxazosin were each found to increase velocity in wild-type larvae. The effects of aceclofenac, amlodipine, and doxazosin were compared to optimal dose of atomoxetine (see FIGS. 3, 4 and 5, respectively). Comparison with Lphn3.1 HOM larvae that were administered the vehicle control, revealed that all doses of aceclofenac, amlodipine, and doxazosin differed significantly from vehicle control and untreated larvae (in a dose dependent manner). These data show that aceclofenac, amlodipine, and doxazosin are effective at reducing ADHD-like phenotypes in Lphn3.1 HOM larvae.

### A Compound According to the Formula (S-AM)

Amlodipine is an L-type calcium channel (LTCC) blocker. LTCCs modulate neuronal excitability, catecholaminergic transmission, cortical plasticity, and neuroinflammation—processes central to ADHD pathophysiology. There has been growing interest in the role of LTCCs, including the Cav1.2 and Cav1.3 subtypes, in ADHD pathophysiology and it is proposed that amlodipine acts within attention/arousal circuits to stabilize dopaminergic and noradrenergic tone, restore D2-autoreceptor feedback, enhance plasticity, and dampen neuroinflammation.

The potential of amlodipine as an ADHD treatment is described in Thorsteinsson, H., et. al, Validation of L-type calcium channel blocker amlodipine as a novel ADHD treatment through cross-species analysis, drug target Mendelian randomization, and clinical evidence from medical records, Neuropsychopharmacology (2025) 50:1145-1155, incorporated herein in its entirety.

Amlodipine has traditionally been viewed as peripherally acting with limited central nervous system (CNS) activity. However, emerging evidence suggests that this view may underestimate its CNS potential. For example, recent preclinical and translational studies have identified that amlodipine not only penetrates the blood-brain barrier (BBB) more effectively than previously thought, but also modulates behavioural domains relevant to ADHD, including hyperactivity, impulsivity and attention. Convergent preclinical findings show phenotype rescue in zebrafish and rat models of ADHD, accompanied by normalization of ADHD-relevant metabolic pathways. Complementary biobank analyses suggest reduced ADHD-like traits among genetically at-risk individuals taking amlodipine. Given enantiomeric pharmacology, S-amlodipine (S-AM), with markedly higher LTCC affinity and fewer off-target liabilities than R-amlodipine (R-AM), emerges as the preferred candidate.

S- and R-forms have distinct pharmacological profiles. S-amlodipine (S-AM) is the active stereoisomer with nearly a 1000-fold higher affinity for LTCCs than R-amlodipine (R-AM). This suggests that S-AM is likely responsible for the LTCC-mediated effects of amlodipine, potentially influencing dopaminergic, noradrenergic, and glutamatergic signaling implicated in ADHD. By contrast, R-AM exhibits minimal calcium-channel activity and mainly engages peripheral mechanisms.

The pharmacological differences lead to distinct outcomes. S-AM has potential for stabilizing DA tone and reducing neuroinflammation, whereas R-AM has been linked to increased nitric oxide (NO) production via the kallikrein-kinin pathway, causing peripheral edema and oxidative stress. In the brain, where redox balance and neurovascular integrity are important, these off-target actions of R-AM could be detrimental. Similarly, by releasing bradykinin and NO through the kallikrein-kinin pathway, R-AM may trigger inflammatory pathways and induce cytokines (e.g. IL-10, IL-6) that could interfere with normal neurotransmission.

Beyond peripheral symptoms, NO signaling can indirectly disrupt DA regulation by promoting oxidative stress or increasing gamma-aminobutyric acid release in the VTA. Previous studies have also suggested that NO inhibits the release of NE. Because ADHD involves dysregulation of DA and NE, these NO-mediated effects of R-AM could be counterproductive. Indeed, clinical trials have shown that formulations containing only S-AM significantly reduce the incidence of peripheral edema, one of the most common side effects associated with racemic amlodipine, while maintaining antihypertensive efficacy.

As described in Thorsteinsson, H., et. al, Validation of L-type calcium channel blocker amlodipine as a novel ADHD treatment through cross-species analysis, drug target Mendelian randomization, and clinical evidence from medical records, Neuropsychopharmacology (2025) 50:1145-1155, amlodipine crosses the blood brain barrier.

### Example 2. Amlodipine Blood Brain Penetration in Zebrafish

### Methods

Adult adgrl3.1−/− zebrafish were maintained at 28.5° C. on a 14:10 light/dark cycle with 10% daily water replacement. Fish were immersed for 30 min in 10 μM amlodipine or vehicle (0.3% DMSO), rinsed in system water, euthanized, and brains extracted. Brain amlodipine was quantified by UPLC coupled to a Q-Exactive HR-MS using four diagnostic transitions (238.6, 294.2, 377.2, 392.1 m/z); at least two transitions were required for confirmation.

### Results

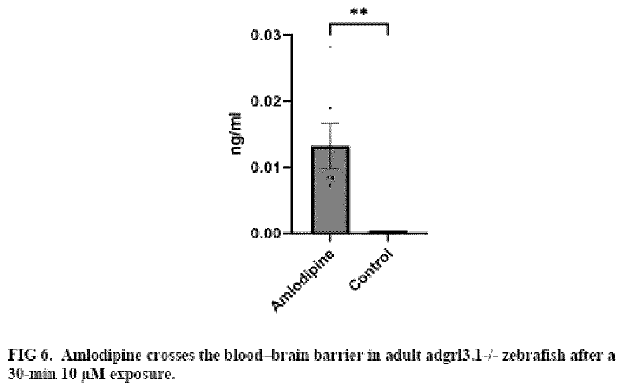

As shown in FIG. 6, amlodipine was detected in exposed brains at 0.013±0.003 ng/mL (n=6). No drug was detected in vehicle brains (n=6) except a single specimen at 9×10⁻⁶ ng/mL, attributed to contamination. No sex differences were observed. These data show that amlodipine crosses the zebrafish blood brain barrier (BBB) following short immersion.

### Example 3. Amlodipine Blood Brain Penetration in Rats

### Methods

Sprague-Dawley rats (n=6; 3 males and 3 females), aged 6-8 weeks were treated with either single or repeated oral administrations of amlodipine at 1 mg/kg or 10 mg/kg (vehicle: 90% acetate buffer, 10% propylene glycol). At 6 h post-dose, blood was collected (lithium-heparin), plasma prepared (2000 g, 5 min, 4° C.), and perfused with saline prior to brain harvest. Brains were homogenized (1:2, w/w) in Ultra-turrax® in ultra-pure water. Plasma and brain were analyzed by LC-MS/MS.

### Results

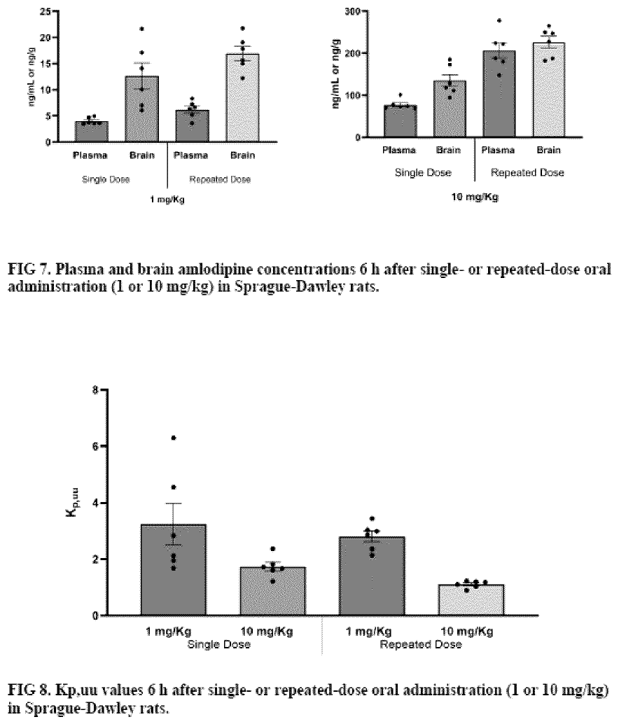

As shown in FIG. 7, no significant sex-based differences were observed (p>0.05). Therefore, the reported concentrations represent combined values across sexes. In the single-dose group, plasma concentrations were 3.99±0.27 ng/ml and 77.68±4.89 ng/ml, while brain concentrations were 12.63±2.48 ng/g and 135.0±14.72 ng/g for the 1 mg/kg and 10 mg/kg doses, respectively. In the repeated-dose group, plasma concentrations increased to 0.21±0.68 ng/ml and 206.40±18.39 ng/ml, with brain concentrations of 16.89±1.37 ng/g and 226.3±14.06 ng/g for the respective doses.

As shown in FIG. 8, K_{p,uu} (unbound brain-to-plasma partition coefficient) values for the single-dose group were 3.24±0.74 (1 mg/kg) and 1.74±0.15 (10 mg/kg), and for the repeated-dose group were 2.81±0.19 (1 mg/kg) and 1.11±0.05 (10 mg/kg). These results demonstrate effective BBB penetration of amlodipine.

### Example 4. Amlodipine Blood Brain Penetration in Mice

### Methods

C3H mice (n=8; 4 males and 4 females), aged 5 months to 1 year, received intraperitoneal amlodipine (10 mg/kg/day) or vehicle (90% acetate buffer, 10% propylene glycol) for 5 days. Blood samples were collected from the orbital sinus under isoflurane anesthesia. After collection, plasma was centrifuged at 1600 g for 10 min at 4° C. Following blood sampling, each mouse was euthanized and the brains were removed and snap-frozen in liquid nitrogen. Plasma and brain samples were analyzed by LC-MS/MS.

### Results

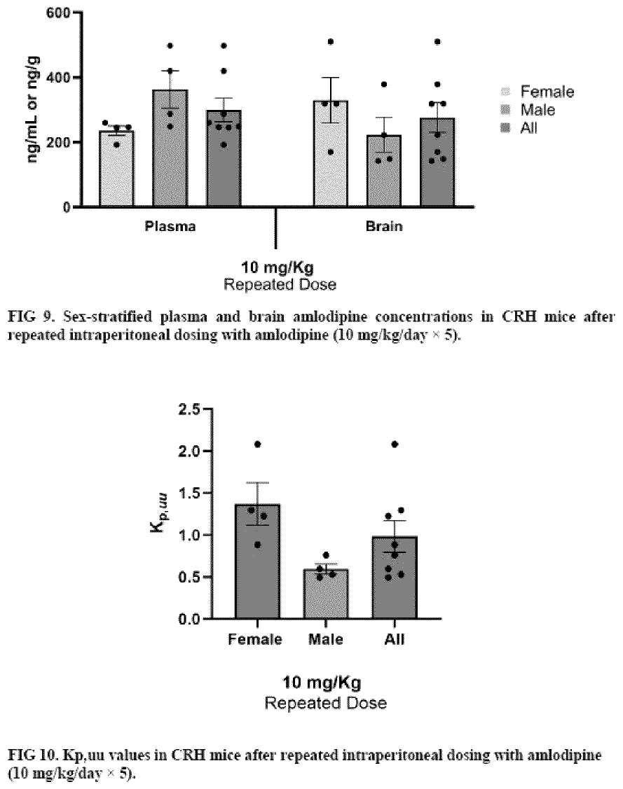

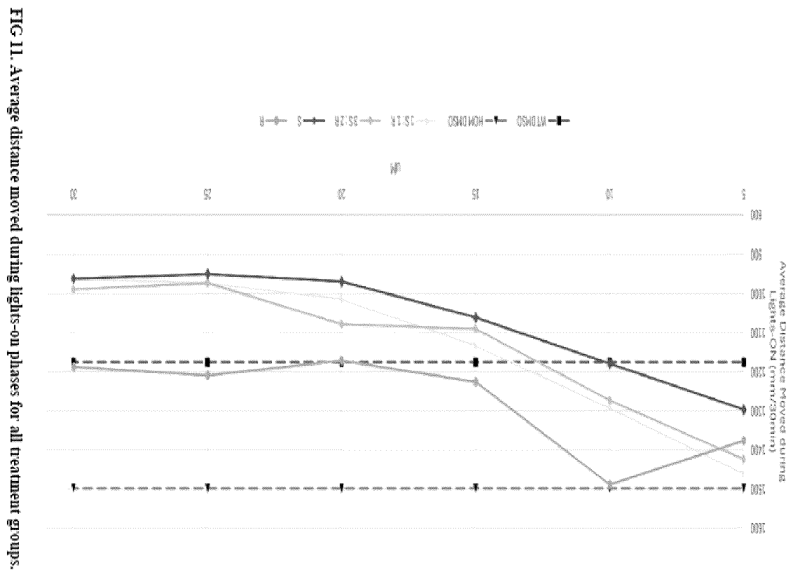

As shown in FIG. 9, females showed mean (±SD) plasma concentrations of 235.54±29.92 ng/mL and brain concentrations of 329.07±139.28 ng/g. Males exhibited higher plasma but lower brain exposure, with 363.14±115.63 ng/mL in plasma and 222.62±110.03 ng/g in brain. Shown in FIG. 10, corresponding K_{p,uu} values were 1.37±0.51 in females and 0.60±0.12 in males. When data were combined across sexes, plasma concentrations were 299.34±103.76 ng/mL and brain concentrations were 275.84±129.38 ng/g, yielding a pooled K_{p,uu} of 0.98±0.54. Overall, amlodipine was detectable in both matrices after repeated dosing. We predict equal brain penetration of S-AM and R-AM when administered independently. See examples 5 and 6, where both enantiomers cause brain and behavioral effects when administered independently.

### Example 5 S-AM and R-AM Phenotype

### Methods

For the analyses of S-AM and R-AM effects on behavior and sleep, X adgrl3.1+/+ zebrafish larvae and X adgrl3.1−/− mutant larvae at 6 days post-fertilization (dpf) were used. S-AM/R-AM proportions of 3:2 and 1:1 were tested. For the 3:2 proportion, S-AM and R-AM were co-administered at 3S:2R, 6S:4R, 9S:6R, 12S:8R, 15S:10R, and 18S:12R μM. For the 1:1 proportion, S-AM and R-AM were co-administered at 2.5S:2.5R, 5S:5R, 7.55:7.5R, 10S:10R, 12.55:12.5R, and 15S:15R. DMSO used as vehicle control. Moreover, S-AM and R-AM were also tested separately at 5, 10, 15, 20, 25, and 30 μM. Fish were maintained at Reykjavik University in a recirculating multi-tank system at 28.5° C. on a 14:10 h light/dark cycle with 10% daily water replacement. All procedures conformed to the National Bioethics Committee of Iceland's regulations (regulation 460/2017).

At 5 dpf (n=70/condition), larvae were randomized to 96-well plates in system water and acclimated for 24 h. Recordings started at 13:00 with lights on and proceeded with alternating 30-min light/dark phases between 13:30 and 18:00. Behavioral endpoint was average distance moved (mm). S-AM and R-AM were dissolved in DMSO. Data were acquired with EthoVision XT and analyzed in GraphPad Prism.

### Results

As shown in FIG. 11, in adgrl3.1−/− larvae, one-way ANOVA for average distance moved showed significant main effects (F(20, 1463)=33.39, p<0.0001). Dunnett's tests identified 19 of the 24 drug-containing combinations with reduced distance moved. All tested concentrations of S-AM, excluding 5 μM, yielded a significant reduction in average distance moved. In contrast, R-AM significantly reduced average distance moved at 15, 20, and 30 μM. For the 3:2 proportion, all concentrations significantly reduced average distance moved, except for the 3S:2R ratio. Similarly, for the 1:1 proportion, all concentrations significantly decreased average distance moved, except for the 2.5S:2.5R ratio.

### Example 6. Brain-Specific Multiomics of S- Vs. R-Amlodipine

To further characterize the stereoselective biological behaviour of the amlodipine enantiomers, we performed an integrated multi-omics analysis comprising transcriptomics, proteomics, and metabolomics on neural tissue exposed to S-amlodipine (S-AM) or R-amlodipine (R-AM).

### Methods

Housing and Fish Care. For metabolomic, proteomic, and transcriptomic analyses, 126 adult AB-WT zebrafish at 17 months post-fertilization (mpf) were used. Fish were maintained at Reykjavik University in a recirculating multi-tank system at 28.5° C. on a 14:10 h light/dark cycle with 10% daily water replacement. All procedures conformed the National Bioethics Committee of Iceland's regulations (regulation 460/2017).

Zebrafish were randomly divided into three treatment groups: S-AM, R-AM, and vehicle control (n=42/group). For metabolomics and transcriptomics, each group comprised 3 biological replicates of 6 pooled brains (18 fish/group). For proteomics, each group consisted of 3 biological replicates of 2 pooled brains (6 fish/group). Pool sizes were determined by the minimum tissue required for each assay.

Drug Treatment and Sample Collection. S-AM and R-AM were initially dissolved in DMSO to make 10 mM stocks, then diluted in system water to 10 μM working solutions. Exposure was performed by static immersion for 4 h. For each condition, nine 500 mL treatment tanks were prepared and five fish were placed in each tank to ensure constant exposure with aeration. After exposure, fish were transferred to clean system water for a 5-min wash to remove residual drug. Randomization was used when pooling fish from different tanks to minimize batch effects. Fish were euthanized by rapid cooling, followed by decapitation. Brains were dissected using sterile micro-dissection tools, rinsed briefly in phosphate-buffered saline to remove blood, blotted dry, placed into labeled 1.5 mL tubes, and frozen at −80° C.

Data Analysis. For brain transcriptomics, gene-level counts estimated from RNA-seq data (Bowtie2/RSEM pipeline) were analysed with DESeq2 (v1.40.2) using a negative-binomial generalized linear model, and Wald tests were used to compute log 2(fold-change) and p-values for pairwise contrasts. Multiple-testing correction was performed with the Benjamini-Hochberg procedure, and genes showing an absolute fold-change ≥1.2 and q-value ≤0.05 were classified as differentially expressed. For label-free DIA proteomics, protein-level quantitative values were compared between groups using Welch's t-test, and proteins with an absolute fold-change ≥1.5 and raw p-value <0.05 in a given comparison were defined as differentially expressed proteins. For untargeted plasma metabolomics, UPLC-MS/MS peak areas processed in metaX after PQN normalization, QC-based LOESS signal correction, and QC-based filtering (coefficient of variation ≤30%) were analysed by univariate fold-change calculations and two-sided t-tests. Metabolites with an absolute fold-change ≥1.2 and p-value <0.05 were considered differentially abundant.

### Results

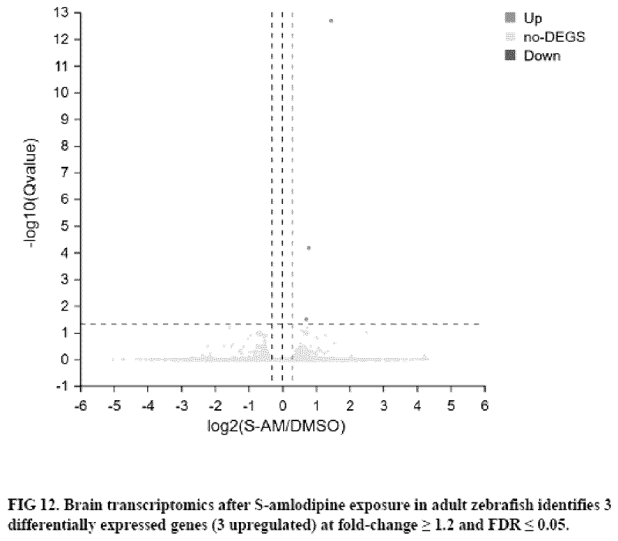

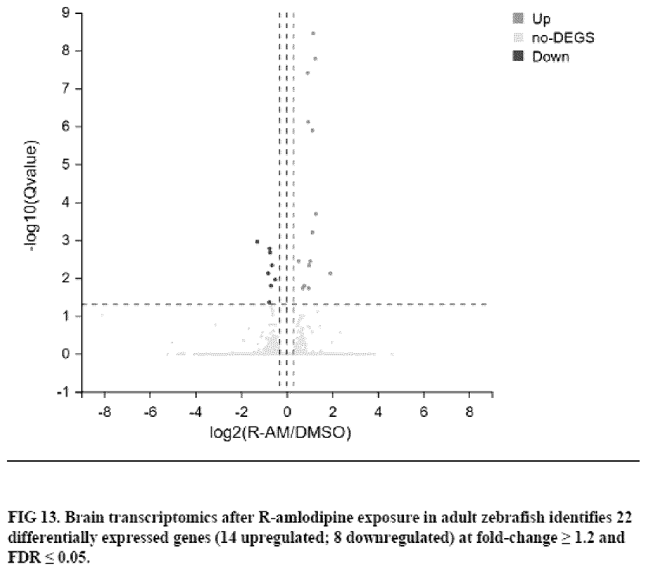

Transcriptomics Analysis. As shown in FIG. 12, transcriptomic profiling of adult zebrafish brains exposed to S-AM or R-AM revealed stereoselective changes in gene expression. Using DESeq2 with an absolute fold-change ≥1.2 and FDR-corrected q-value ≤0.05, S-AM-treated fish showed 3 differentially expressed genes (DEGs) relative to vehicle controls, all of which (klf11a, tubb5, and pim3) were upregulated. In contrast, R-AM treatment yielded 22 DEGs compared to vehicle, with 14 genes upregulated and 8 downregulated, shown in FIG. 13.

To facilitate translational interpretation, differentially expressed zebrafish genes were mapped to their human orthologs, and, unless otherwise stated, gene symbols in this section refer to the corresponding human orthologs. Within the S-AM response, selective upregulation of the zebrafish ortholog of human PIM3, together with KLF11 and TUBB5, points to a focused but neuron-relevant transcriptional effect. PIM3 is a serine/threonine kinase of a family implicated in activity-dependent signalling, neuronal survival and synaptic plasticity, processes that are central to cognitive domains such as attention and working memory. Thus, the S-AM-specific induction of PIM3 is consistent with activation of a pro-plasticity and CNS-relevant kinase pathway.

By contrast, the R-AM transcriptomic signature is enriched for zebrafish genes corresponding to human enzymes and regulators of cholesterol biosynthesis, sterol metabolism and lipid handling (including HMGCR, HMGCS1, MVDA, MSMO1, SQLE, LSS, ELOVL2, HSD17B7, LPIN1 and ABCG4), as well as additional transcription- and chromatin-associated factors such as CBX7 and DDX5. This pattern is consistent with preferential modulation of an SREBP-linked mevalonate and lipid-metabolism programme in brain tissue, rather than engagement of neuronal signalling pathways directly related to synaptic plasticity and cognition. Taken together, these stereoselective transcriptomic patterns differentiate the enantiomers at the molecular level and are aligned with S-AM, but not R-AM, acting as the CNS-active form.

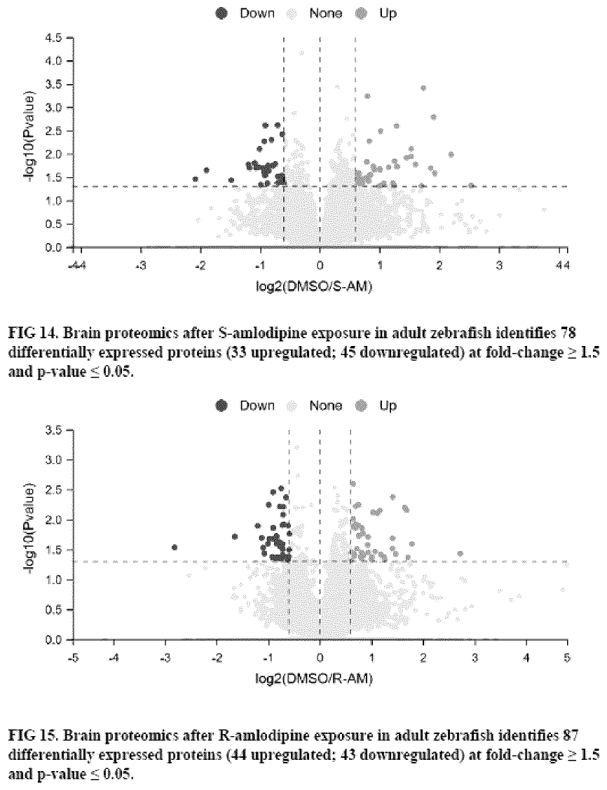

Proteomic Analysis. As shown in FIG. 14, label-free proteomic profiling of adult zebrafish brains confirmed enantiomer-specific protein signatures. Zebrafish treated with S-AM showed 78 differentially expressed proteins compared to controls. Of these, 33 were upregulated and 45 downregulated in the S-AM group. In contrast, R-AM treatment resulted in 87 differentially expressed proteins relative to controls, shown in FIG. 15. Of these, 44 were upregulated and 43 downregulated in the R-AM group.

In the S-AM group, the regulated proteins include several with established roles in neuronal function, plasticity, and lipid handling. S-AM increased proteins corresponding to NDRG4, MAPK7, NTNG2, and ABCD1, alongside multiple ribosomal and RNA-processing factors. NDRG4 is required for normal spatial learning and memory and supports brain-derived neurotrophic factor levels. MAPK7/ERK5 promotes neuronal survival and adult neurogenesis, and its activation enhances performance in hippocampus-dependent learning tasks. NTNG2 encodes the presynaptic adhesion molecule netrin-G2, which organizes excitatory synapses and whose loss-of-function variants are linked to neurodevelopmental disorders with cognitive impairment. ABCD1 is a peroxisomal transporter for very-long-chain fatty acids; loss of ABCD1 causes a demyelinating neurodegenerative disorder, highlighting the importance of adequate ABCD1-mediated lipid handling for CNS integrity. Together, these protein changes are consistent with engagement of pathways that support neuronal survival, synaptic connectivity and myelin-related lipid homeostasis, processes that are central to attention, working memory and other cognitive domains.

In contrast, the R-AM proteomic signature is dominated by proteins involved in cellular metabolism, lipid handling, and stress adaptation, with less direct enrichment for canonical plasticity-related proteins. R-AM altered multiple metabolic enzymes and lipid-associated proteins, including PGAM1A, AK1, ATP5H, CYB5R3, ACBD7 and APOC2. CYB5R3 and related reductases participate in mitochondrial redox reactions that modulate lipid and cholesterol metabolism as well as reactive oxygen species production, while APOC2 is a cofactor for lipoprotein lipase and a key regulator of triglyceride-rich lipoprotein metabolism. R-AM also changed the abundance of an ALOX5 ortholog, which generates leukotrienes and is implicated in inflammatory and neuroinflammatory processes. In parallel, R-AM upregulated several nuclear and chromatin-associated proteins (CUX1, RFX1, PHF14, SETDB1B, EXOSC4, POLE3, SREK1) and membrane/cytoskeletal regulators (DNM3A, RAB11B, GRM2, GRK6, PCDH11, KCNH8, COL4A2, SEPT8, ANKRD10B, FMNL3), indicating broad remodeling of transcriptional control, membrane structure and vesicle trafficking rather than a focused synaptic-plasticity program.

A further feature of the R-AM profile is the coordinated modulation of stress-response and genome-maintenance factors. R-AM increased HSPA13, PRDX5 and GPX4B, while decreasing NMNAT1 and altering RIF1. HSPA13 is an ER-associated HSP70 family chaperone that regulates protein translocation and contributes to endoplasmic reticulum and cytosolic proteostasis. PRDX5 and GPX4 are key antioxidant and ferroptosis-regulating enzymes that protect cells from lipid peroxides and oxidative damage, particularly in mitochondria, whereas NMNAT1 is a nuclear NAD⁺-synthetic enzyme whose loss disrupts neuronal metabolism and accelerates neurodegeneration in the retina. The combined regulation of these factors by R-AM is consistent with activation of oxidative-stress, ER-stress and DNA-maintenance responses, indicative of a metabolic-stress adaptation rather than a primary enhancement of neuronal plasticity.

Taken together, the proteomic data reinforce the stereoselective biology observed at the transcriptomic level. S-AM produces a relatively focused protein-level response that converges on molecules supporting neuronal survival, synaptic organization and brain lipid homeostasis. In contrast, R-AM induces a broader reprogramming of metabolic, lipid, and stress-response pathways, together with chromatin and structural remodeling, pointing to a predominantly metabolic and stress-adaptation profile in brain tissue that is less directly linked to pro-plasticity mechanisms.

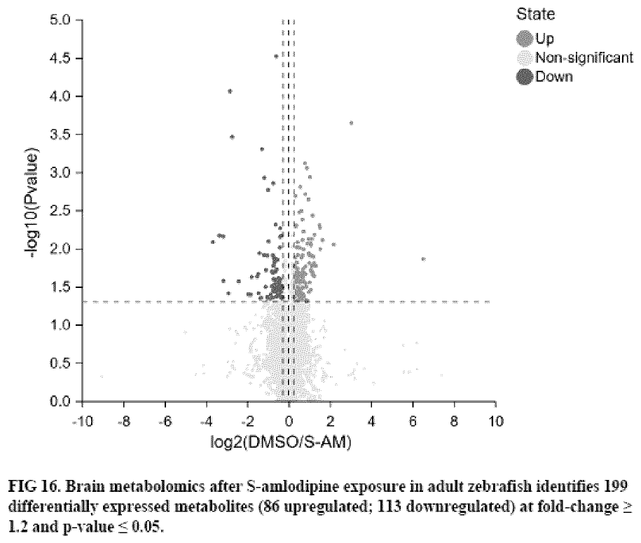

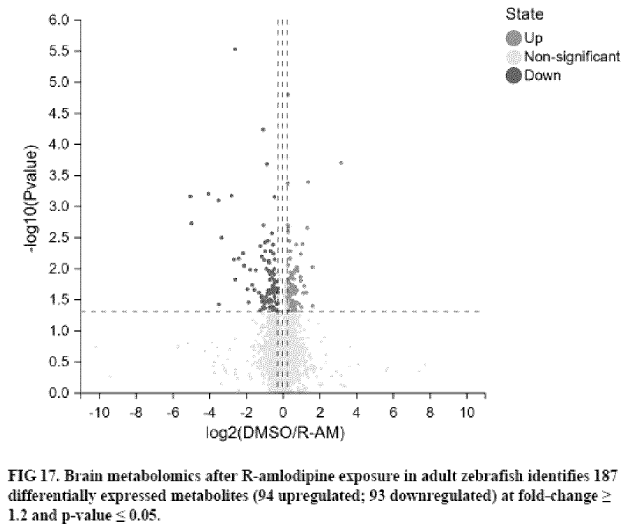

Metabolomic Analysis. As shown in FIG. 16, metabolomics revealed 199 differentially expressed metabolites in zebrafish treated with S-AM compared to controls. Of these, 86 were upregulated and 113 were downregulated in the S-AM group. In contrast, R-AM treatment resulted in 187 differentially expressed metabolites relative to controls, shown in FIG. 17. Of these, 94 were upregulated and 93 downregulated in the R-AM group. Direct comparison of the two enantiomers identified 166 metabolites that differed between S-AM and R-AM (71 relatively enriched in S-AM and 95 enriched in R-AM), confirming a robust stereoselective metabolic response.

In the S-AM group, the differential metabolites formed a broad and multi-pathway signature encompassing amino-acid derivatives, tricarboxylic-acid cycle intermediates, lipid mediators, bile-acid conjugates, sphingolipids, and acylcarnitines. Examples include increased oxoglutaric acid, 2-arachidonoylglycerol (2-AG), D-sphingosine, and very-long-chain acylcarnitines such as tricosanoylcarnitine and 6-hydroxyheptanoylcarnitine, together with altered phospholipid precursors. This pattern is consistent with coordinated support of mitochondrial energy metabolism and membrane lipid remodelling in the nervous system, in line with the known importance of carnitine/acylcarnitine pathways and lipid metabolism for brain energy supply, myelin integrity, and cognitive function. Notably, 2-AG is a key endocannabinoid that mediates on-demand retrograde suppression of synaptic transmission and promotes synaptic plasticity and hippocampal neurogenesis. Within this framework, the S-AM metabolomic profile is compatible with a CNS-active, neurophysiologically relevant mechanism that could help normalize network excitability and attentional control.

S-AM also modulated several redox-related metabolites, including ophthalmic acid, cysteine-S-sulfate, sulfur-containing small molecules, glutathione conjugates, and nitrated signalling lipids. Ophthalmic acid is a structural analogue of glutathione whose levels track glutathione turnover and oxidative-stress responses across diverse systems, whereas FAD-dependent and nitro-fatty-acid pathways are increasingly recognized as mediators of anti-inflammatory and cytoprotective signalling. Taken together, the S-AM metabolic signature suggests an adaptive adjustment of energy and redox homeostasis in parallel with neuromodulatory lipid signalling.

By contrast, R-AM induced a more lipid-centric and stress-biased metabolic profile. The most prominent changes involved bile-acid and sterol-related metabolites, with decreases in several taurine- and glycine-conjugated bile acids, including tauro-β-muricholic acid, tauroursodeoxycholic acid (TUDCA), taurolithocholic acid 3-sulfate, taurodeoxycholic acid, and glycocholic acid. R-AM therefore appears to down-tune circulating levels of bile acids such as TUDCA that have well-documented neuroprotective, anti-apoptotic, and anti-inflammatory actions in experimental models of neurodegeneration and CNS injury. This bile-acid signature aligns with the mevalonate/cholesterol-focused transcriptomic and proteomic programs observed for R-AM and suggests a shift towards altered gut-liver-brain bile-acid signalling that is less favourable for neuronal resilience.

R-AM also produced stronger evidence of lipid peroxidation and detoxification activity. Long-chain ceramides and very-long-chain acylcarnitines such as nervonyl carnitine were preferentially increased, together with multiple phospholipid, lysophospholipid, and glycolipid species. Dysregulation of ceramide and acylcarnitine metabolism is closely linked to mitochondrial dysfunction, myelin lipid imbalance, and cognitive deficits, indicating that this R-AM signature reflects stress-driven remodelling of lipid homeostasis rather than an activity-linked neurometabolic adjustment. In addition, R-AM selectively elevated F2-isoprostane species such as 8,12-iso-iPF2α-VI, which are widely accepted as sensitive and specific in vivo biomarkers of oxidative lipid damage and have been associated with neurodegenerative and cognitive disorders. These findings indicate that R-AM preferentially engages a bile-acid, oxidative-stress, and detoxification-oriented metabolic programme.

Taken together, S-AM induces a broad, multi-pathway metabolic signature characterized by enhanced endocannabinoid/sphingolipid signalling, support of mitochondrial and acylcarnitine-dependent energy metabolism, and engagement of adaptive redox pathways, a pattern that is mechanistically compatible with CNS-targeted, pro-plasticity effects. In contrast, R-AM drives a more constrained, lipid- and bile-acid dominated profile marked by depletion of neuroprotective bile acids, accumulation of long-chain ceramides and acylcarnitine, and increased oxidative stress. These stereoselective metabolomic differences further differentiate the enantiomers and support S-AM as the preferred CNS-active candidate for therapeutic development.

1. A compound according to formula (S-AM): 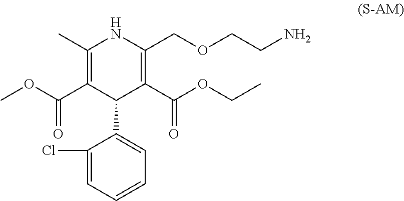 [SMILES: CCOC(=O)C1=C(COCCN)NC(C)=C(C(=O)OC)[C@H]1c1ccccc1Cl]
or a pharmaceutically acceptable salt or solvate thereof, for use in the treatment or prophylaxis of Attention Deficit/Hyperactivity Disorder (ADHD).

2. The compound for use according to claim 1, wherein the compound is administered in a dose of about 0.1 mg to 5 mg (excluding the mass of any counterion or solvent).

3. The compound for use according to claim 1, wherein a dose of the compound is administered as a single daily dose of about 0.1 mg, 1 mg, 2 mg, 3 mg, 4 mg, or 5 mg (excluding the mass of any counterion or solvent).

4. The compound for use according to claim 1, wherein a dose of the compound is administered at a dose of 0.1 mg, 1 mg, 2 mg, 3 mg, 4 mg or 5 mg (excluding the mass of any counterion or solvent), two, three, or four times a day.

5. A method for the treatment or prophylaxis of Attention Deficit/Hyperactivity Disorder (ADHD) comprising administering to a patient a dose of a compound according to formula (S-AM).

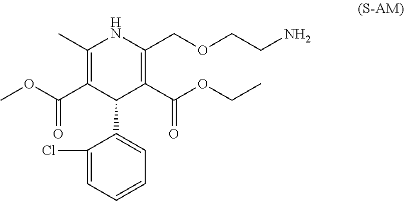 [SMILES: CCOC(=O)C1=C(COCCN)NC(C)=C(C(=O)OC)[C@H]1c1ccccc1Cl]

6. The method of claim 5, wherein the compound is administered in a dose of about 0.1 mg to 5 mg (excluding the mass of any counterion or solvent).

7. The method of claim 5, wherein a dose of the compound is administered as a single daily dose of 0.1 mg, 1 mg, 2 mg, 3 mg, 4 mg, or 5 mg (excluding the mass of any counterion or solvent).

8. The method of claim 5, wherein a dose of the compound is administered at a dose of 0.1 mg, 1 mg, 2 mg, 3 mg, 4 mg, or 5 mg (excluding the mass of any counterion or solvent), two, three or four times a day.

9. The method of claim 5, wherein the compound is administered in a pharmaceutical composition.

10. The method of claim 5, wherein the compound is administered simultaneously, sequentially, or separately with one or more further therapeutic interventions.

11. The method of claim 10, where the one or more further therapeutic interventions are selected from a dietetic intervention, a psychological intervention, and a pharmacological intervention.

12. The method of claim 11, wherein the pharmacological intervention is selected from methylphenidate, dexamphetamine, lisdexamfetamine, atomoxetine, and guanfacine.

13. The method of claim 5, wherein the ADHD is selected from “impulsive type/hyperactive type” ADHD, “inattentive type” ADHD, and “combined type” ADHD.

14. The method of claim 5, wherein the patient is known or suspected of having ADHD or known or suspected of being at risk of developing ADHD.

15. The method of claim 14, wherein the patient known or suspected of having ADHD, or known or suspected of being at risk of developing ADHD, has a sign or symptom of another condition selected from depression, anxiety disorder, oppositional defiant disorder (ODD), conduct disorder, a sleep problem, autism spectrum disorder (ASD), bipolar disorder, epilepsy, Tourette's syndrome, and a learning difficulty.

16. The method of claim 15, wherein the sleep problem is sleep onset insomnia.

17. The method of claim 15, wherein the learning difficulty is dyslexia.

18. The method of claim 15, wherein the compound is administered to a patient for whom one or more established ADHD treatments have been ineffective at reducing one or more of the symptoms of ADHD or induced intolerable adverse effects.

19. The method of claim 18, wherein the one or more established ADHD treatments are selected from methylphenidate, dexamphetamine, lisdexamfetamine, atomoxetine, viloxazine, guanfacine, and clonidine.

20. The method of claim 18, wherein the intolerable adverse effects are selected from impaired sleep, lack of appetite, increased blood pressure, stunted growth, disruption of circadian rhythm, and neurotoxicity.

21. A kit comprising a compound according to formula (S-AM): 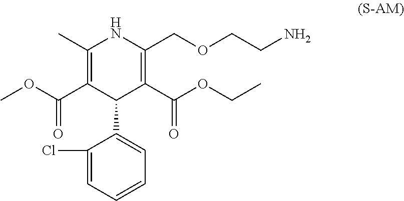 [SMILES: CCOC(=O)C1=C(COCCN)NC(C)=C(C(=O)OC)[C@H]1c1ccccc1Cl]
or a pharmaceutically acceptable salt or solvate thereof, and one or more further pharmacological interventions, wherein the one or more further pharmacological interventions is selected from methylphenidate, amphetamine, lisdexamfetamine, atomoxetine, viloxazine, clonidine, and guanfacine.

22. The kit of claim 21, wherein the amphetamine is dexamphetamine.

23. A method for the treatment or prophylaxis of ADHD comprising administering a kit of claim 21.
